(19) United States
(12) Patent Application Publication
(10) Pub. No.: US 2023/0226346 A1
(43) Pub. Date: Jul. 20, 2023

(54) RECHARGEABLE STIMULATION APPLIANCE

(71) Applicants: Jeff Bennett, Maple Grove, MN (US); Alan Carlson, St. Paul, MN (US); Christopher Narveson, Minneapolis, MN (US); Jon Lawson, Cottage Grove, MN (US)

(72) Inventors: Jeff Bennett, Maple Grove, MN (US); Alan Carlson, St. Paul, MN (US); Christopher Narveson, Minneapolis, MN (US); Jon Lawson, Cottage Grove, MN (US)

(21) Appl. No.: 18/092,895

(22) Filed: Jan. 3, 2023

Related U.S. Application Data
(60) Provisional application No. 63/296,130, filed on Jan. 3, 2022.

(51) Int. Cl. A61N 1/04; A61N 1/36

(52) U.S. Cl. CPC A61N 1/0496; A61N 1/36014

(57) ABSTRACT

An electrical stimulation therapy appliance, system, and method for neuromodulation and treatment of various neurological conditions, including but not limited to sexual dysfunctions like premature ejaculation or decreased orgasm intensity in males, and persistent genital arousal or decreased arousal in females. The electrical stimulation therapy appliance, system, and method including a detachable control housing that can be recharged or reprogramed. The control housing being removably connectable to a disposable patch.

### PRIORITY

This patent application claims priority to and the benefit of U.S. Provisional Pat. Application No. 63/296,130, filed Jan. 3, 2022, which is incorporated fully herein by reference.

### FIELD

The present invention relates generally to the treatment of stimulation appliances for treating various conditions, including pelvic health conditions such as a sexual dysfunction like premature ejaculation or decreased orgasm intensity in males, and persistent genital arousal or decreased arousal in females. More particularly, the present invention pertains to an apparatus or appliance that is rechargeable and reusable.

### BACKGROUND

A number of devices and methods are available for enabling those with health conditions such as a sexual dysfunction like premature ejaculation or decreased orgasm intensity in males, and persistent genital arousal or decreased arousal in females. These devices and method are generally either applied to the surface of the genitals, in the form of a pharmacological cream, inserted into the vaginal or anal canal, or are implanted within or proximate to the genitals in order to deliver an electrical pulse to a nerve that innervates the genitals.

Generally speaking, the methods that are available, or which have been described, include the use of various constriction devices. These devices, like the one described in U.S. Pat. No. 5,921,914 have been used for centuries and are typically applied around a base of a penis to constrict it. The constriction causes the penis to stay erect and prevents the flow of semen. The problem with these constriction devices is that blood is prevented from flowing out of the penis. This permits a blood pooling effect that can causes the temperature of the penis to drop causing it to feel cold. This can be unpleasant for the person suffering from premature ejaculation and their sexual partner.

When topical compounds and drugs failed to provide an adequate solution, medical device companies developed various electrical stimulation devices that stimulate the nerves of the penis in an attempt to prevent premature ejaculation. For instance, in U.S. Pat. No. 7,328,069 to Gerber, Medtronic has developed a device that is implantable into an abdomen of a patient with leads extending into a patient’s pelvic cavity to stimulate the pudendal nerve. The problem of these implantable devices is that they carry the shortcomings of all the complications associated with surgery, including but not limited infection. Others developed electrical stimulation devices that did not have to be implanted but would rather be placed over the penis. These devices, like the one described in U.S. Pat. No. 9,017,244 to Chiu, used cuffs or condom shaped devices that would deliver electrical stimulation to the penis.

In all of the devices available or described a need has remained for an improved device and method for treating sexual dysfunction. A device and method of treating a sexual dysfunction such as premature ejaculation or decreased orgasm intensity in males, and persistent genital arousal or decreased arousal, in females that doesn’t negatively impact the experience of both the sufferer and partner, nor requires implantation would be very desirable. Additionally, a device and method of treatment that can be recharged and/or reused. A device or appliance that is capable of being recharged and reused eliminates the concern that the device or appliance may not work when needed. Further, it reduces the amount of waste being generated.

### SUMMARY

The present invention is a treatment for pelvic health dysfunctions or sexual dysfunctions such as, for example, premature ejaculation or decreased orgasm intensity in males, and persistent genital arousal or decreased arousal in females. Premature ejaculation, for instance, is a condition impacting up to 30% of men worldwide.

The present invention is a rechargeable and reusable appliance that is intended to sense an anatomical physiological change such as an approaching ejaculatory event and then to apply a stimulation therapy to transdermally stimulate or confuse a nerve, such as the pudendal nerve or muscles, to stop, cease, or change an anatomical physiological event such as an ejaculatory event. For the control of ejaculation, the appliances, devices and methods described herein stimulate (e.g., electrical stimulation), and thus inhibit, the nerve pathway between the penis and the brain in order to delay ejaculation until the male or female desires to have an ejaculation occur. The appliances, devices, and methods described herein are able to be programmed for the treatment of a particular medical condition.

In one example embodiment of the invention a cuff, ring, patch, clip, or similar type of device is removably secured to a portion of the penis or to another anatomical location effective to treating a condition such as premature ejaculation. The device may be secured to a portion of the penis or the perineum, or another anatomical location to deliver the treatment or therapy. The device includes one or more sensors that are capable of sensing an anatomical event such as an approaching ejaculatory event or event, such as a sneeze, that may cause incontinence. The sensors are capable of detecting any type of physiological, chemical, or electrical phenomenon related to the anatomical event. For example, the sensors are able to detect an increase or change in the girth of the penis or a state of an electrical potential generated by the penis.

The cuff, patch, or clip can also comprise a wave producing mechanism generated by an ultrasound crystal, to create waves that interfere with various nerves of the penis or assist in transmitting the compound through the dermis. The waves are transmitted at a frequency that interferes with the signal pathway thus delaying an ejaculatory event. In another example embodiment of the invention, the cuff may contain other elements (electrical, mechanical, chemical or magnetic) that when activated would also interfere with nerve signals from the penis to delay ejaculation.

The present invention may also include a wired or wireless controller that controls the delivery of the treatment. For example, the controller can be activated via a dedicated device or a smart phone, watch, or similar device that someone would wear or hold. Anyone having control of the controller would be able to activate or deactivate the therapy device such as a cuff, ring or other support structure. When deactivated, the therapy device would not interfere with nerve signals transmitted between the anatomy and physiological event to be treated. When activated, however, the therapy device would cause ultra nerve confusion and prevent an ejaculatory event. The level of nerve signal confusion may be controlled by the person holding or wearing the controller so that the timing of a desired ejaculation can be controlled by either party.

Another novel aspect of the invention is the detachable control assembly that is detachable from the appliance. The control assembly can be removed for charging and then reattached to the appliance to power and control the therapy. The control assembly can also be replaced with another or different control assembly that is programmed for another applying another or different therapy.

The control assembly includes a control housing that is attachable to a base attached to or formed as part of the appliance. The control housing includes one or more contacts that are configured to mate or contact one or more contacts of the base. The contacts of the housing and the base are configured to communicate and transmit power and the therapy from the control housing to the appliance that is positioned on the user. The control housing also includes one or more securing mechanisms formed therein or coupled to it. The securing mechanism is configured to mate with a portion of the base, whereby a user is able to attach and detach the control housing from the base.

The above summary is not intended to limit the scope of the invention, or describe each embodiment, aspect, implementation, feature or advantage of the invention. The detailed technology and preferred embodiments for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

The device of the present invention (hereinafter, “device”) is described in detail below with reference to examples of preferred embodiments illustrated in the accompanying drawings. The invention may be developed in multiple embodiments, with all sharing key design features such as use of “securing mechanism” (which may include be spring-loaded or biased tensioners as shown in the drawings or other securing mechanisms) to secure the control housing to the base.

FIG. 1A is a perspective view of a control assembly comprising a control housing coupled to a base of the therapy appliance according to an example embodiment.

FIG. 1B is a perspective view of a control assembly positioned on the appliance according to an example embodiment.

FIG. 1C is a perspective view of a control assembly positioned on the appliance according to an example embodiment.

FIG. 2A is perspective view of contacts of the control housing of the therapy appliance according to an example embodiment.

FIG. 2B is perspective view of the control housing of the therapy appliance according to an example embodiment.

FIG. 3A is perspective view of a securing mechanism of the control housing of the therapy appliance according to an example embodiment.

FIG. 3B is perspective view of a securing mechanism of the control housing of the therapy appliance according to an example embodiment.

FIG. 4A is a perspective view of a base of a control assembly according to an example embodiment of the invention.

FIG. 4B is a perspective view of a base of a control assembly according to an example embodiment of the invention.

FIG. 5A is a perspective view of a base of a control assembly according to an example embodiment of the invention.

FIG. 5B is a perspective view of a base of a control assembly according to an example embodiment of the invention.

FIG. 6A is a cross sectional view showing the securing mechanism of the control assembly according to an example embodiment of the invention.

FIG. 6B is a cross sectional view showing the contact communication of the control assembly according to an example embodiment of the invention.

FIG. 6C is a cross sectional view showing the contact communication of the control assembly according to an example embodiment of the invention.

This following specification, which refers to the above figures, includes references to “one embodiment” or “an embodiment,” “an example embodiment,” and the like. The appearances of phrases do not necessarily refer to the same embodiment. Particular features, structures, or characteristics may be combined in any manner consistent with this disclosure.

The term “comprising” is to be considered open-ended. As used in the appended claims, this term does not foreclose additional structure or steps. The terms “First,” “Second,” and the like, are used as labels for nouns that they precede, and do not imply any type of ordering (e.g., spatial, temporal, logical, etc.).

### DETAILED DESCRIPTION

In the following descriptions, the present invention will be explained with reference to various embodiments or exemplary embodiments. Nevertheless, these embodiments are not intended to limit the present invention to any specific example, environment, application, or particular implementation described herein. Therefore, descriptions of these example embodiments are only provided for the purpose of illustration rather than to limit the present invention.

Dimensions and relative proportions of components are merely example embodiments and can be varied unless specifically limited in a given claim. Thus, the dimensions can be varied without departing from the scope of the invention.

The present invention illustrates appliances, devices, systems, and methods for controlling a therapy delivery device, such as an appliance for treating sexual dysfunctions such as premature ejaculation or diminished ejaculation intensity. While the invention is particularly advantageous for patients suffering from sexual dysfunction such as premature ejaculation, it may also be used by anyone that desires to delay or alter sexual emissions. The present invention may also be used as a means of desensitizing an organ or a sexual organ for the purpose of altering sexual activity or emissions. The present invention may have applications in humans as well as veterinary applications. Although the present invention is described as being applicable to males, it should be appreciated that the present invention can also be used to treat a female’s pelvic health disorder, including but not limited to, sexual dysfunction and incontinence.

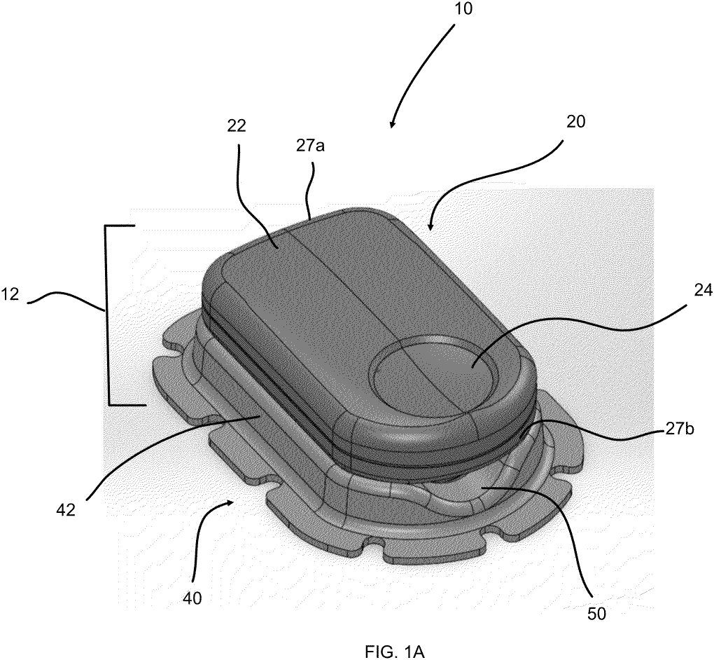

The present invention is directed to a therapy appliance 10, that generally comprises a control assembly 12 coupled to and in operative communication with a therapy interface or patch 14 (see FIG. 1B). The therapy interface or patch 14 is configured to be placed on a therapy location of a user to apply a particular therapy to the therapy location. While the therapy is applied to the therapy location it should be understood that the therapy may travel beyond the specific therapy location to treat distant tissues, muscles, and nerves.

As will be discussed in more detail, one or more portions of the control assembly 12 are detachable or removable from other portions of the control assembly 12 or the therapy interface or patch 14 to be recharged, reprogrammed, and/or to connect to a different therapy interface or patch 14, or to connect a different control assembly 12 to the therapy interface or patch 14.

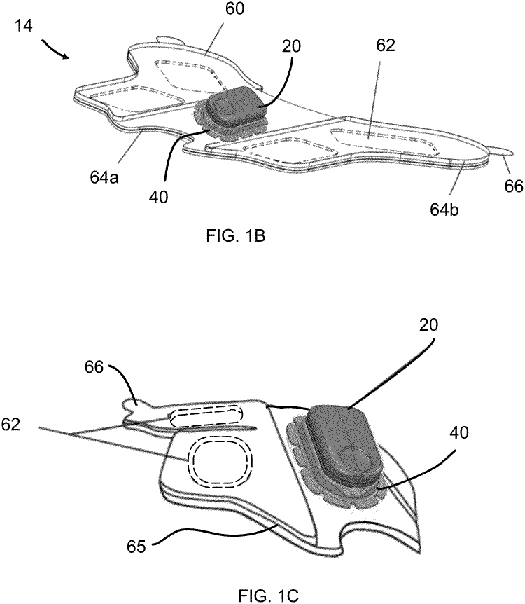

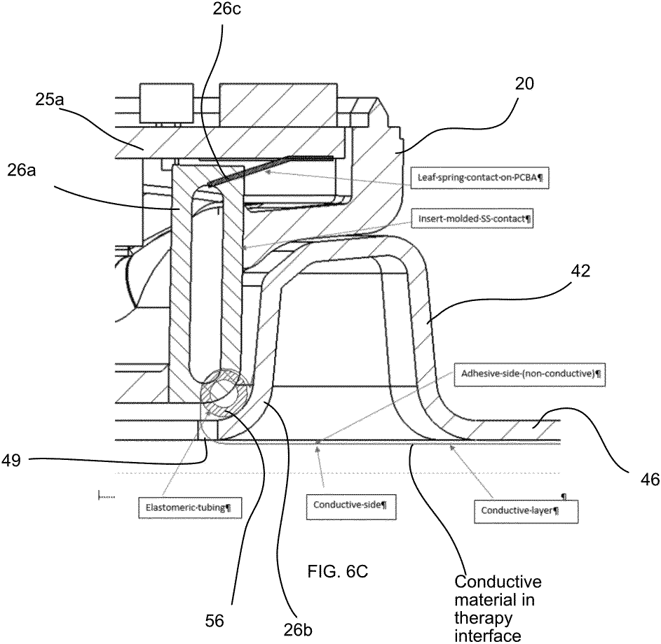

In one example embodiment, the control assembly 12 comprises a control housing 20 that is connectable to and detachable from a base 40 that is operatively coupled to a portion of the therapy interface or patch 14. Control housing 20 comprises an outer shell or casing 22 (see FIG. 2A) and an interior 23 (see FIG. 6A). The interior 23 of the control housing 20 is configured to house a control module 25a and a power supply 25b. In one example embodiment of the invention, as illustrated in FIGS. 6A and 6B, the outer shell or casing 22 may comprise one or more shell portions 22a and 22b coupled or attached together. A seal member may be positioned between the shell portions 22a and 22b to seal the interior 23 from contaminations such as water and perspiration.

As illustrated in FIG. 1A, control housing 20 includes an actuator 24 that is operatively coupled to the control module 25a contained within interior 23 of the control housing 20. Actuator 24 can be operatively disposed on or in outer shell 22 in a location that is convenient for a user to access. Actuator 24 is controlled by a user to control various functions of control assembly 12. For example, to power on/off appliance 10, control the therapy (e.g., intensity, frequency, duration), change therapies, and the like. Actuator 24 may comprise any type of controller, including but not limited to depressible switches, touch sensitive switches, slide switches, and the like. Appliance 10 and its control module 25a may also be controlled by a remote controller 25c such as a phone, Ipad, smart watch, or any other remote-control device that can communicate with the control module 25a.

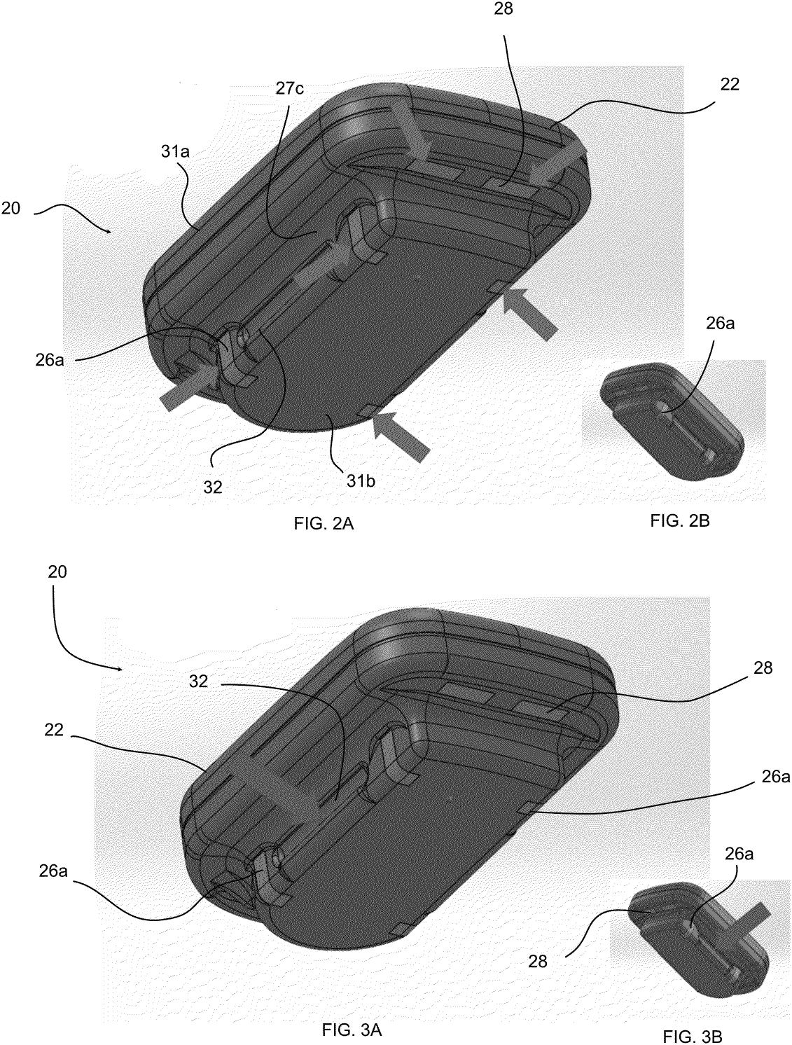

As illustrated in FIGS. 2A-3B, outer shell 22 of control housing 20 comprises one or more therapy or control contacts 26a that are configured to operatively communication with control contacts 26b on a portion of the base 40. As particularly illustrated in FIG. 2A, control contacts 26a are positioned on a lower portion of outer shell 22 and, as illustrated in FIGS. 6A and 6B, extend into interior 23 of outer shell 22 where they are operatively coupled a portion of at least the control module 25a. Control contacts 26a may comprise any conductive or electrically conductive material that is operatively coupled to at least the control module 25a.

Control housing 20 may also include one or more charging contacts 28 that are at least operatively coupled to power supply 25b. As illustrated in FIGS. 2A-3B, charging contacts 28 may be disposed proximate to one or more ends 27a and 27b of control housing 20. Control housing 20 can be configured to mate with a charging base that is able to charge power supply 25b of control housing 20. While a charging contact 28 has been described, other methods of charging are also contemplated. For instance, alternative charging methods include a charging port, remote charging, USB charging, and the like.

As illustrated in FIG. 6A, outer shell 22 of control housing 20 and a portion of base 40 can also comprise one or more securing mechanisms 30 for securing or temporarily securing control housing 20 to a portion of base 40. In the example embodiment illustrated in FIGS. 2A-3B, securing mechanism 30 comprises a detent or recess 32 extending into a side portion 27c of outer shell 22. Detent or recess 32 may be generally parallel to a long axis of control housing 20 and may extend between the pair of control contacts 26a. Detent or recess 32 can also comprise more than one detent or recess 32

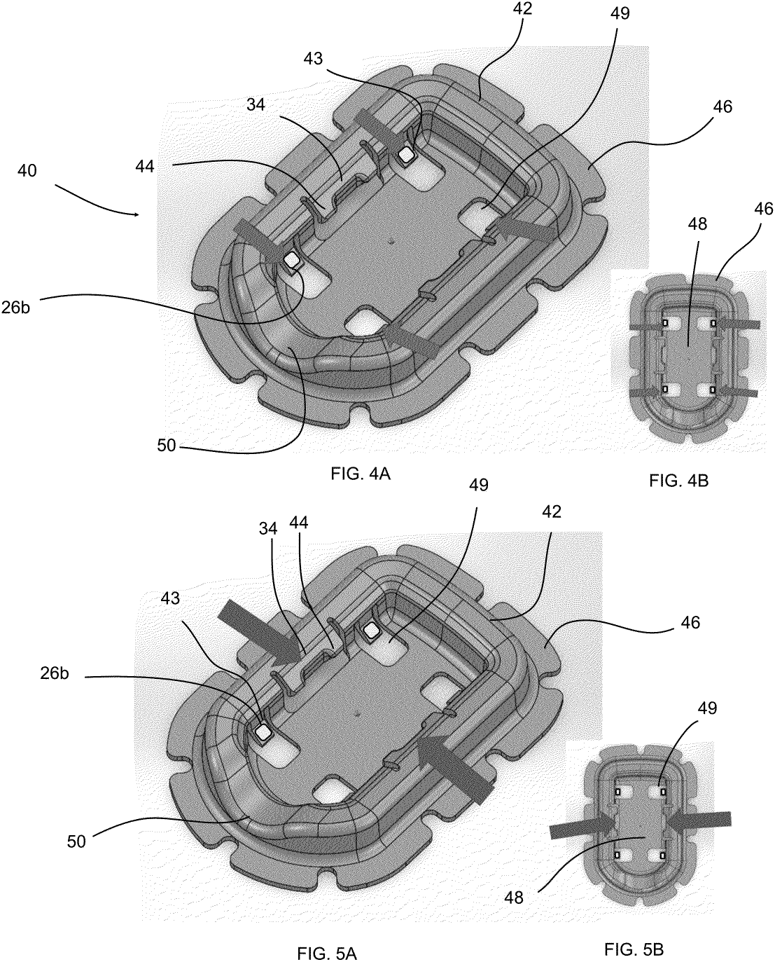

As illustrated FIGS. 4A and 4B, an example embodiment of the present invention includes base 40 having a peripheral wall or lip 42 that is configured to mate with control housing 20. Base 40 includes one or more control contacts 26b that contact or mate with control contacts 26a of control housing 20. Control contacts 26a and 26b transmit the energy or therapy from control module 25a to the therapy interface or patch 14. An example embodiment of control contacts 26b of base 40 include one or more arms 43 formed in or coupled to the base 40. Arms 43 may comprise one or more conductive materials disposed thereon that are configured to mate with control contacts 26a of control housing 20.

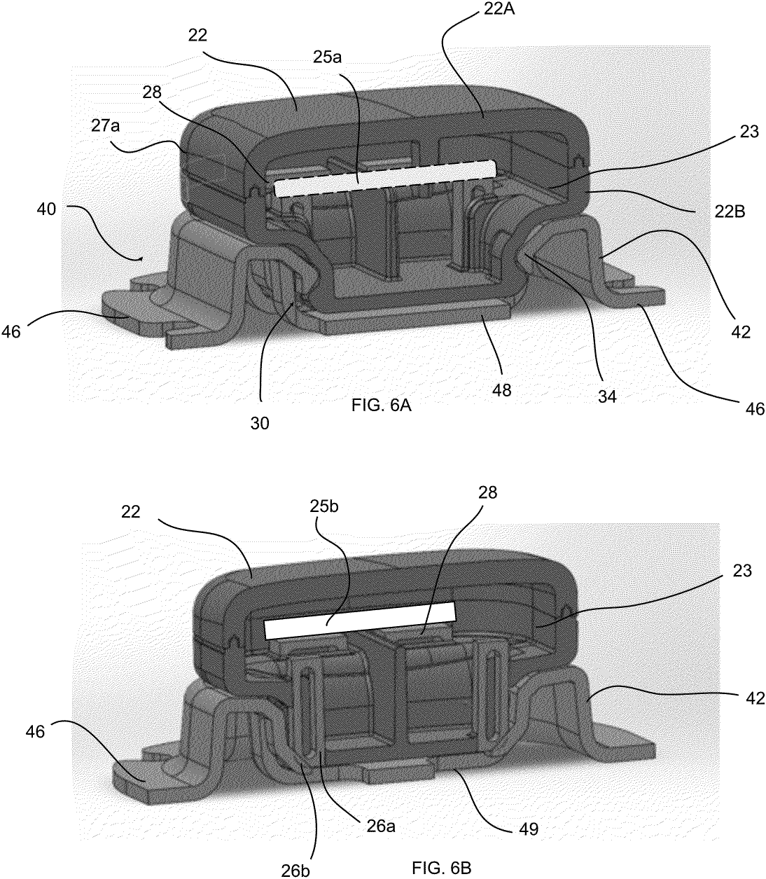

Base 40 can also include portions of securing mechanisms 30. In one example embodiment, as illustrated in FIGS. 4A-5B, securing mechanism 30 comprise one or more ridges 34 formed into or coupled to base 40 to mate with the detent 32 of the control housing 20. As particularly illustrated in FIGS. 4A-5B, ridges 34 on base 40 can extend from a portion of lip 42 of base 40. Ridges 34 can also comprise one or more projections 44 that extend into detent or recess 32 of control housing 20. Control housing 20 can comprise at least one detent 32 on each of its lateral sides 27c that are configured to receive or mate with ridges 34 and/or projections 44 of base 40.

As particularly illustrated in FIGS. 2A-3B, control housing 20 can comprise an upper portion 31a and a lower portion 31b with upper portion 31a having a wider cross section than lower portion 31b. The smaller cross section of lower portion 31b enables it to mate into an interior or recess of base 40. The larger cross section of upper portion 31a, which contains actuator 24, is sized to allow a lower surface of upper portion 31a to rest upon an upper surface or top of lip 42. Lip 42 acts as a backstop to upper portion 31a, which allows, enables, or creates a generally firm and stable surface for upper portion 31a, whereby a user can easily control or depress actuator 24.

As illustrated in FIGS. 4A-5B, base 40 can comprise one or more tabs 46 that extend away from a portion of base 40. In one example embodiment, tabs 46 extend outwardly away from a lower portion of a peripheral wall or lip 42 of base 40. Tabs 46 extend generally parallel to the surface of the therapy interface patch 14 and provide stability and a coupling surface of base 40 to the therapy interface or patch 14. Base 40 can also comprise a bottom wall 48 having one or more openings 49 extending therethrough. Openings 49 can be positioned generally proximate to control contacts 26b. Bottom wall 48 provides additional support to base 40 while, as illustrated in FIG. 6C, openings 49 provide a conduit for the conductive material of the control contacts 26b to be coupled to or in operative communication with a conductive material extending through therapy interface or patch 14.

Peripheral wall or lip 42 of base 30 can also comprise a depression 50 formed in a portion of peripheral wall or lip 42 whereby a user may insert their finger or a tool to separate the control housing 20 from base 40. Securing mechanisms 30, in this configuration, may be generally a friction fit that can be overcome by pulling control housing 20 away from base 40. While not shown, other securing mechanisms are also contemplated herein, including but not limited to magnets, snaps, buttons, and the like.

Turning to FIG. 6C, control module 25a can comprise a pulse generator capable of generating a therapy stimulation such as a pulse of electrical energy. Control contact 26c is operatively coupled between control module 25a and control contact 26a in control housing 20. Control contact 26c can comprise a spring that can be operatively coupled to actuator 24, whereby a user is able to control therapy appliance 10 by depressing or activating actuator 24 and completing or breaking a circuit between control contacts 26a and 26c. In this manner, control module 25a, under the power of the power supply 25b, controls a transmission or application of the therapy to the user or therapy location.

In another example embodiment of the invention, as illustrated in FIG. 6C, a conductive carrier 56, configured to receive a conductive material of therapy interface or patch 14, can be positioned in base 40 to interface with control contacts 26a of control housing 20. Conductive carrier 56 can comprise an elastomeric tubing with the conductive material of the therapy interface or patch 14 wound about it. Conductive carrier 56 can be deformable upon contact with control housing 20 and its control contacts 26a.

The therapy interface or patch 14 can comprise one or more of the devices taught and disclosed in U.S. Pat. Application Nos. 16/618,099, filed Jun. 13, 2018; 17/421,161 filed Jul. 9, 2021; and PCT Pat. Application No. PCT/US2020/067234 filed Dec. 28, 2020; including all of their priority documents, the entirety of which are all incorporated herein in their entireties by reference.

In one example embodiment, as illustrated in FIGS. 1B and 1C, therapy interface patch 14 comprises a patch 60 having one or more electrode pads 62 (broken lines) that are operatively in communication with control module 25a of the control assembly 12. Patch 60 includes or can comprise one or more insulating layers 64a and 64b that house or protect the conductive material of the therapy interface 14 or patch 60. A conductive adhesive 65 can be applied to patch 60 to allow or enable a user to comfortably place patch 60 to the therapy location. Protective film 66 can be removed prior to use and reapplied after application of the therapy. Protective film 66 will prevent conductive adhesive 65 from being contaminated between uses. Conductive adhesive 65 can be replaced by simply peeling it off of patch 60 and applying a new adhesive 65.

Control assembly 12 is configured to securely attach the pulse generator (control module 25a) to electrode pads 62 in a proper orientation, keeps the control module 52 in place during use, and provides low-resistance electrical connections between the circuitry of the pulse generator 25a and the conductive layer or material in electrode pads 62. The design of control assembly 12 achieves a low-resistance connection as well as providing sufficient contact pressure via mechanically compliant (“spring-loaded”) elements.

As briefly mentioned above, in other example embodiments, one or more magnets can be used to establish polarization and provide sufficient force for physical retention of control housing 20 (housing the pulse generator) and in some cases, contact pressure for electrical connections, in base 40. In another example embodiment, snap features are used by mating undercuts between a receptacle on therapy interface or patch 14 and control housing 20 housing the pulse generator. These securing features or mechanisms 30 can be manufactured from injection molds, cantilever snap features, thermoformed undercuts, metal clips, and the like.

There can also be several other types of connections between control housing 20 and base 40. Including the following:
A vertical compression connection or contact deflection from control housing 20 (housing the pulse generator). This mechanical attachment provides the contact pressure required for making a good electrical connection. A conductive gasket material (Z-axis and XYZ types or film-over-foam EMC gasket) can also be employed.
Vertically-acting metal stampings or springs and conductive tape can also be used to connect to electrode pads 62 to conductive layer 65.
A foam component under the electrode pad’s 62 and/or conductive layer 65 to lift it up and generate an interference with solid contact in the pulse generator or control module 25a.
Lateral Opposing Type - contact pressures from opposing L-R sides and independent of mechanical retention forces can be used.
Side-acting metal stampings and conductive tape to connect to conductive layer 65 can be used.
Film-over-Foam EMC gasket can also be used.
Electrical Pad conductive layer adhered to compliant interference features/components in receptacle assembly.
Metal stampings for electrical contact also interface with physical features in the solid contact on the pulse generator which provide physical retention of the pulse generator into the receptacle.

While the invention has been described in connection with what is presently considered to be the most practical and preferred embodiments, it will be apparent to those of ordinary skill in the art that the invention is not to be limited to the disclosed embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products. Moreover, features or aspects of various example embodiments may be mixed and matched (even if such combination is not explicitly described herein) without departing from the scope of the invention.

What is claimed is:

1. A therapy appliance configured to deliver electric stimulation therapy to a therapy location, the therapy appliance comprising:
an adherable patch comprising a lower surface and an upper surface, the upper surface comprising one or more electrodes configured to deliver the therapy;
a base member coupled to a portion of the adherable patch, the base member having one or more base contacts in operative communication with the one or more electrodes;
a control housing removably mateable with the base member, the control housing having a control module and a power supply configured to generate the therapy, the control housing having one or more housing contacts that are connectable to or mateable with the one or more base contacts; and
wherein the control module generates the therapy that is transmitted through the housing contacts and base contacts, which delivers it to the one or more electrodes that deliver it to the therapy location.

2. The therapy appliance of claim 1, further comprising at least one securing mechanism coupled to extending between the base member and the control housing, wherein the at least one securing mechanism releasably secures the control housing to a portion of the base member.

3. The therapy appliance of claim 2, wherein the at least one securing mechanism comprises at least one protection extending away from a portion of the base member, wherein the at least one projection is removably extendable into at least one detent formed in a portion of the control housing to secure it to the base member.

4. The therapy appliance of claim 2, wherein the at least one securing mechanism comprises a first magnet portion disposed on a portion of the base member and a second magnet portion disposed on a portion of the control housing.

5. The therapy appliance of claim 1, wherein the control housing comprises a wider upper portion and a narrower lower portion, wherein the narrower lower portion is removably positionable in a recess formed in the base member.

6. The therapy appliance of claim 1, further comprising an actuator disposed to a portion of the control housing and in operative communication with the control module.

7. The therapy appliance of claim 6, wherein the base comprises a peripheral wall configured to support a portion of the control housing when coupled together.

8. The therapy appliance of claim 1, wherein the at least one or more base contacts comprises an elastomeric conductive member contactable and deformable for the one or more housing contacts.

9. The therapy appliance of claim 8, wherein the elastomeric conductive member comprises a elastomeric tube having a conductive material wound about an exterior surface of the elastomeric tube.

10. The therapy appliance of claim 1 further comprising a charger connectable to the one or more housing contacts of the control housing to charge the power supply.

11. A therapy application system configured to deliver electric stimulation therapy to a therapy location, the system comprising:
an adherable patch comprising a lower surface and an upper surface, the upper surface comprising one or more electrodes configured to deliver the therapy;
a base member coupled to the lower surface of the adherable patch, the base member having one or more base contacts in operative communication with the one or more electrodes;
a control housing having matable shell portions defining an interior configured to house a control module and a power supply that are configured to generate the therapy, the control housing having one or more housing contacts extending through at least one of the shell portions and connectable to or mateable with the one or more base contacts;
an actuator disposed on a portion of the control housing and in operative communication with at least the control module;and
wherein the control housing is detachably connectable to a portion of the base member and when connected the control module generates the therapy that is transmitted through the housing contacts and base contacts, which delivers the therapy to the one or more electrodes.

12. The therapy application system of claim 11 further comprising at least one first securing mechanism portion coupled a portion of the control housing and at least one second securing mechanism portion coupled to a portion of the base member, wherein the at least one first securing mechanism portion and the at least one second securing portion are releasably mateable to secure the control housing to a portion of the base member.

13. The therapy application system of claim 12, wherein the at least one first securing mechanism portion comprises at least one protection member and the at least one second securing mechanism portion comprises at least one detent formed in a portion of the control housing, wherein the at least one projection member is removably insertable into at least one detent to secure it to the base member.

14. The therapy application system of claim 12, wherein the at least one securing mechanism comprises a first magnet portion disposed on a portion of the base member and a second magnet portion disposed on a portion of the control housing.

15. The therapy application system of claim 11, wherein the control housing comprises a wider upper portion and a narrower lower portion, wherein the narrower lower portion is removably positionable in a recess formed in the base member.

16. The therapy application system of claim 15, wherein the base member comprises a peripheral wall configured to contact and support the wider upper portion of the control housing when coupled together, the peripheral wall stabilizing the control housing when actuating the actuator.

17. The therapy application system of claim 11, wherein the at least one or more base contacts comprises an elastomeric conductive member contactable and deformable by the one or more housing contacts.

18. The therapy application system of claim 17, wherein the elastomeric conductive member comprises a elastomeric tube having a conductive material wound about an exterior surface of the elastomeric tube and connected to a portion of the electrodes.

19. The therapy application system of claim 11 further comprising a charger connectable to the one or more housing contacts of the control housing to charge the power supply housed in the interior of the control housing.

20. A method of applying electric stimulation therapy to a therapy location, the method comprising the steps of:
providing an adherable patch comprising a lower surface and an upper surface, the upper surface comprising one or more electrodes configured to deliver the electric therapy;
providing a base member coupled to the lower surface of the adherable patch, the base member having one or more base contacts in operative communication with the one or more electrodes;
providing a control housing comprising an actuator in operative communication with a control module and a power supply contained within an interior of the control housing, one or more housing contacts in operative communication with the control module, wherein the control module is configured to generate the electric therapy that travels through the one or more housing contacts and the one or more base contacts; and
wherein the control housing is detachably connectable to a portion of the base member and when connected the control module generates the therapy that is transmitted through the housing contacts and base contacts, which delivers the therapy to the one or more electrodes.
